(19) United States
(12) Patent Application Publication
(10) Pub. No.: US 2019/0231317 A1
(43) Pub. Date: Aug. 1, 2019

(54) ULTRASOUND SCANNING SYSTEM

(71) Applicant: Massachusetts Institute of Technology, Cambridge, MA (US)

(72) Inventors: Brian W. Anthony, Cambridge, MA (US); Athena Yeh Huang, Palo Alto, CA (US)

(21) Appl. No.: 16/235,978

(22) Filed: Dec. 28, 2018

Related U.S. Application Data
(60) Provisional application No. 62/611,333, filed on Dec. 28, 2017.

(51) Int. Cl. A61B 8/08; A61B 8/00; A61B 5/00; G01N 29/22

(52) U.S. Cl. CPC A61B 8/485; A61B 8/4472; A61B 5/0053; G01N 29/226; A61B 8/4209; A61B 8/429; A61B 8/463; A61B 5/6843

(57) ABSTRACT

A force-sensing ultrasound imaging device uses multiple force sensors to capture the spatial distribution of a contact force between the imaging device and a target surface. In another aspect, the device may provide an external force stimulus in order to facilitate synchronization of different hardware systems that independently acquire force sensor data and ultrasound transducer data.

### CROSS-REFERENCE TO RELATED APPLICATIONS

This application claims the benefit of U.S. Provisional Patent Application No. 62/611,333, filed Dec. 28, 2017, the entire content of which is hereby incorporated by reference.

### STATEMENT OF GOVERNMENT INTEREST

This invention was made with government support under Grant No. U01 EB018813 awarded by the National Institutes of Health. The government has certain rights in the invention.

### FIELD OF THE INVENTION

The invention generally relates to ultrasound imaging.

### BACKGROUND

While medical ultrasound images can be quickly and cheaply obtained from a handheld ultrasound imaging device, this type of imaging generally suffers from a lack of accurate information concerning the conditions under which the scan was captured. As a result, two-dimensional ultrasound images from handheld probes are generally limited in use to a qualitative evaluation of the imaged tissue. There remains a need for an ultrasound imaging device that captures additional context to facilitate improved imaging and quantitative analysis.

### SUMMARY

A force-sensing ultrasound imaging device uses multiple force sensors to capture the spatial distribution of a contact force between the imaging device and a target surface. In another aspect, the device may provide an external force stimulus in order to facilitate synchronization of different hardware systems that independently acquire force sensor data and ultrasound transducer data. In another aspect, an acquisition-state enhanced ultrasound imaging device can use multiple sensors to supplement ultrasound data by capturing additional contextual information about acquisition states. This acquisition state data may be used to improve image quality, quantitative accuracy, repeatability and so forth.

### BRIEF DESCRIPTION OF THE FIGURES

The invention and the following detailed description of certain embodiments thereof may be understood by reference to the following figures, wherein similar reference characters denote similar elements throughout the several views.

FIG. 1 is a perspective view of a handheld ultrasound probe control device.

FIG. 2 is a schematic view of a handheld ultrasound probe.

FIG. 3 is a flowchart of a process for force-controlled acquisition of ultrasound images.

FIG. 4 shows a lumped parameter model of the mechanical system of a probe as described herein.

FIG. 5 is a flowchart depicting operating modes of a force-controlled ultrasound probe.

FIG. 6 shows a process for ultrasound image processing.

FIG. 7 shows an exploded view of an ultrasound probe with a distributed load sensor.

FIG. 8 shows a cross section of an ultrasound probe with a distributed load sensor.

FIG. 9 shows a method for using an ultrasound transducer with distributed force sensing.

FIG. 10 shows a device for applying a force to an object.

FIG. 11 shows a method for synchronizing force data and ultrasound data from a force-sensing ultrasound probe.

FIG. 12 shows a generalized ultrasound workflow using acquisition states.

### DETAILED DESCRIPTION

The embodiments will now be described more fully hereinafter with reference to the accompanying figures, in which preferred embodiments are shown. This invention may, however, be embodied in many different forms and should not be construed as limited to the illustrated embodiments set forth herein. Rather, these illustrated embodiments are provided so that this disclosure will convey the scope of the invention to those skilled in the art.

All documents mentioned herein are hereby incorporated in their entirety by reference. References to items in the singular should be understood to include items in the plural, and vice versa, unless explicitly stated otherwise or clear from the text. Grammatical conjunctions are intended to express any and all disjunctive and conjunctive combinations of conjoined clauses, sentences, words, and the like, unless otherwise stated or clear from the context. Thus, the term “or” should generally be understood to mean “and/or” and so forth.

Recitation of ranges of values herein are not intended to be limiting, referring instead individually to any and all values falling within the range, unless otherwise indicated herein, and each separate value within such a range is incorporated into the specification as if it were individually recited herein. The word “about,” when accompanying a numerical value, is to be construed as indicating a deviation as would be appreciated by one of ordinary skill in the art to operate satisfactorily for an intended purpose. Ranges of values and/or numeric values are provided herein as examples only, and do not constitute a limitation on the scope of the described embodiments. The use of any and all examples, or exemplary language (“e.g.,” “such as,” or the like) provided herein, is intended merely to better illuminate the embodiments and does not pose a limitation on the scope of the embodiments. No language in the specification should be construed as indicating any unclaimed element as essential to the practice of the embodiments.

In the following description, like reference characters designate like or corresponding parts throughout the figures. Additionally, in the following description, it is understood that terms such as “front,” “back,” “first,” “second” and the like, are words of convenience and are not to be construed as limiting terms.

The techniques described herein enable real-time measurement and control of the contact force between an ultrasound probe and a target, such as a patient's body. More generally, the techniques described herein enable real-time measurement of acquisition states and use of this acquisition state data to improve the quality, utility, improvability, and quantitative accuracy of ultrasound data and images reconstructed therefrom.

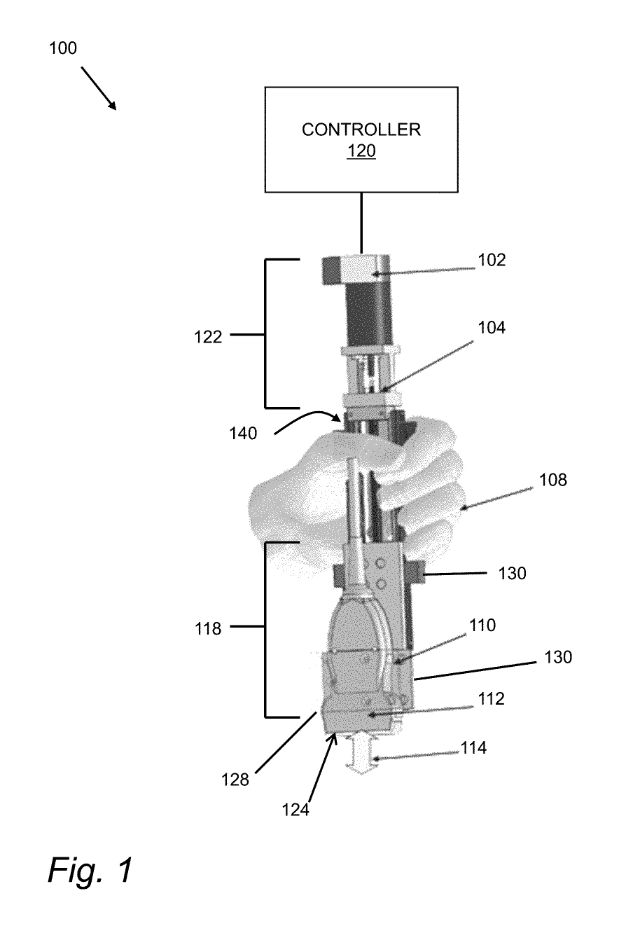

FIG. 1 is a perspective view of a handheld ultrasound probe control device. The device 100 may include a frame 118 adapted to receive a probe 112, a linear drive system 122 that translates the frame 118 along an actuation axis 114, a sensor 110 such as a force sensor, a torque sensor, position sensor 142 or some combination of these, and a controller 120. The device may also include a linear drive system that positions the frame 118 in other directions.

The probe 112 can be of any known type or construction. The probe 112 may, for example, include a handheld ultrasound probe used for medical imaging or the like. More generally, the probe 112 may include any contact scanner or other device that can be employed in a manner that benefits from the systems and methods described herein. Thus, one advantage of the device 100 is that a standard off-the-shelf ultrasound medical probe can be retrofitted for use as a force-controlled ultrasound in a relatively inexpensive way, i.e., by mounting the probe 112 in the frame 118. Medical ultrasound devices come in a variety of shapes and sizes, and the frame 118 and other components may be adapted for a particular size/shape of the probe 112, or may be adapted to accommodate varying sizes and/or shapes. In another aspect, the probe 112 may be integrated into the frame 118 or otherwise permanently affixed to (or in) the frame 118.

In general, a probe 112, such as an ultrasound probe, includes an ultrasound transducer 124. The construction of suitable ultrasound transducers is generally well known. In one aspect, an ultrasound transducer includes piezoelectric crystals or similar means to generate ultrasound waves and/or detect incident ultrasound. More generally, any suitable arrangement for transmitting and/or receiving ultrasound may be used as the ultrasound transducer 124 in the embodiments described herein. Still more generally, other transceiving mechanisms or transducers may also or instead be used to support imaging modalities other than ultrasound.

The frame 118 may include any substantially rigid structure that receives and holds the probe 112 in a fixed position and orientation relative to the frame 118. The frame 118 may include an opening that allows an ultrasound transducer 124 of the probe 112 to contact a patient's skin or other surface through which ultrasound images are to be obtained. Although FIG. 1 shows the probe 112 held within the frame 118 between two plates (a front plate 128 and a larger plate 130, where the front plate 128 is bolted to the larger plate 130 on the frame 118) arranged to surround a handheld ultrasound probe and securely affix the probe to the frame 118, any suitable technique may also or instead be employed to secure the probe 112 in a fixed relationship to the frame 118. For example, the probe 112 may be secured with a press fit, hooks, screws, anchors, adhesives, magnets, or the like, or any combination of these and other fasteners. More generally, the frame 118 may include any structure or combination of structures suitable for securely retaining the probe 112 in a fixed positional relationship relative to the probe 112, for example, including but not limited to a sleeve, pocket, or the like.

In one aspect, the frame 118 may be adapted for handheld use, and more particularly adapted for gripping by a technician in the same orientation as a conventional ultrasound probe. Without limitation, this may include a trunk 140 or the like for gripping by a user that extends axially away from the ultrasound transducer 124 and generally normal to the contact surface of the transducer 124. Stated alternatively, the trunk 140 may extend substantially parallel to the actuation axis 114 and be shaped and sized for gripping by a human hand. In this manner, the trunk 140 may be gripped by a user in the same manner and orientation as a typical handheld ultrasound probe. The linear drive system 122 may advantageously be axially aligned with the trunk 140 to permit a more compact design consistent with handheld use. That is, a ballscrew or similar linear actuator may be aligned to pass through the trunk 140 without diminishing or otherwise adversely affecting the range of linear actuation.

The linear drive system 122 may be mounted on the device 100 and may include a control input electronically coupled to the controller 120. The linear drive system 122 may be configured to translate the probe 112 along an actuation axis 114 in response to a control signal from the controller 120 to the control input of the linear drive system 122. Additionally, the linear drive system 122 may include a position sensor 142 to provide position information characterizing a position of the probe 112 along the actuation axis 114. The position may, for example, be a position relative to one or more travel limits of the linear drive system 122. Although the linear drive system 122 is depicted by way of example as a motor 102 and a linear actuator 104, any system capable of linearly moving the probe 112 can be employed. For example, the linear drive system 122 can include a mechanical actuator, hydraulic actuator, pneumatic actuator, piezoelectric actuator, electro-mechanical actuator, linear motor, telescoping linear actuator, ballscrew-driven linear actuator, or the like. More generally, any actuator or combination of actuators suitable for use within a grippable handheld form factor such as the trunk 140 may be suitably employed as the linear drive system 122. Moreover, the linear drive system 122 may include gears, motors, rails, or the like, in addition to, or in lieu of, linear actuators. In some implementations, the linear drive system 122 is configured to have a low backlash (e.g., less than 3 μm) or no backlash in order to improve positional accuracy and repeatability.

The ability of the probe 112 to travel along the actuation axis 114 permits the technician some flexibility in hand placement while using the device 100. In some implementations, the probe 112 can travel up to six centimeters along the actuation axis 114, although greater or lesser ranges of travel may be readily accommodated with suitable modifications to the linear actuator 104 and other components of the device 100.

The motor 102 may be electrically coupled to the controller 120 and mechanically coupled in a fixed positional relationship to the linear actuator 104. The motor 102 may be configured to drive the linear actuator 104 in response to control signals from the controller 120, as described more fully herein. The motor 102 can include a servo motor, a DC stepper motor, a hydraulic pump, a pneumatic pump, or the like.

The sensor 110, which may include a force sensor and/or a torque sensor, may be mechanically coupled to the frame 118, such as in a fixed positional relationship to sense forces/torques applied to the frame 118. The sensor 110 may also be electronically coupled to the controller 120, and configured to sense a contact force between the probe 112 and a target surface (also referred to herein simply as a “target”) such as a body from which ultrasound images are to be captured. As depicted, the sensor 110 may be positioned between the probe 112 and the back plate of the frame 118. Other deployments of the sensor 110 are possible as long as the sensor 110 is capable of detecting the contact force (for a force sensor) between the probe 112 and the target surface. Embodiments of the sensor 110 may also or instead include a multi-axis force/torque sensor, a plurality of separate force and/or torque sensors, or the like.

The force sensor may be mechanically coupled to the ultrasound probe 112 and configured to obtain a pressure applied by the ultrasound probe 112 to the skin surface or a target 136. The force sensor may include a pressure transducer coupled to the ultrasound probe 112 and configured to sense an instantaneous contact force between the handheld ultrasound probe 112 and the skin.

The sensor 110 can provide output in any known form, and generally provides a signal indicative of forces and/or torques applied to the sensor 110. For example, the sensor 110 can produce analog output such as a voltage or current proportional to the force or torque detected. Alternatively, the sensor 110 may produce digital output indicative of the force or torque detected. Moreover, digital-to-analog or analog-to-digital converters (not shown) can be deployed at any point between the sensors and other components to convert between these modes. Similarly, the sensor 110 may provide radio signals (e.g., for wireless configurations), optical signals, or any other suitable output that can characterize forces and/or torques for use in the device 100 described herein.

The controller 120 may generally include processing circuitry to control operation of the device 100 as described herein. The controller 120 may receive signals from the sensor 110 indicative of force/torque and from the position sensor 142 of the linear drive system 122 indicative of the position of the probe 112 relative to the travel end points, and may generate a control signal to a control input of the linear drive system 122 (or directly to the linear actuator 104) for maintaining a given contact force between the ultrasound probe 112 and the target, as described more fully herein. The controller 120 may include analog or digital circuitry, computer program code stored in a non-transitory computer-readable storage medium, and the like. Embodiments of the controller 120 may employ pure force control, impedance control, contact force-determined position control, and the like.

The controller 120 may be configured with preset limits relating to operational parameters such as force, torque, velocity, acceleration, position, current, and the like, so as to immediately cut power from the linear drive system 122 when any of these operational parameters exceed the preset limits. In some implementations, these preset limits are determined based on the fragility of the target. For example, one set of preset limits may be selected where the target is a healthy human abdomen, another set of preset limits may be selected where the target is a human abdomen of an appendicitis patient, etc. In addition, preset limits for operational parameters may be adjusted to accommodate discontinuities such as initial surface contact or termination of an ultrasound scan (by breaking contact with a target surface).

In some implementations, the device 100 includes a servo-motor-driven ballscrew linear actuator comprising a MAXON servo motor (EC-Max #272768) (motor 102) driving an NSK MONOCARRIER compact ballscrew actuator (linear actuator 104). a MINI40 six-axis force/torque sensor (sensor 110) from ATI INDUSTRIAL AUTOMATION, which simultaneously monitors all three force and all three torque axes, may be mounted to the carriage of the actuator, and a TERASON 5 MHz ultrasound transducer (ultrasound transducer 124) may be mounted to the force/torque sensor.

The vector from a geometric origin of the sensor 110 to an endpoint at the probe 124 that contacts a patient can be used to map the forces and torques at the sensor 110 into the contact forces and torques seen at the probe/patient interface. In some implementations, it is possible to maintain a set contact force with a mean error of less than about 0.2% and, in a closed-loop system, maintain a desired contact force with a mean steady state error of about 2.1%, and attain at least about 20 Newtons of contact force. More generally, in an embodiment a steady state error of less than 3% was achieved for applied forces ranging from one to seven Newtons.

Other sensors (indicated generically as a second sensor 138) may be included without departing from the scope of this invention. For example, a second sensor 138 such as an orientation sensor or the like may be included, which may be operable to independently detect at least one of a position and an orientation of the device 100, such as to track location and/or orientation of the device 100 before, during, and after use. This data may help to further characterize operation of the device 100. A second sensor 138 such as a range or proximity detector may be employed to anticipate an approaching contact surface and place the device 100 in a state to begin an ultrasound scan. For example, a proximity sensor may be operable to detect a proximity of the ultrasound transducer 124 to a subject (e.g., the target surface). One or more inertial sensors may be included in the device 100. Suitable inertial sensors include, for example, inertial sensors based on MEMS technology such as accelerometers and gyroscopes, or any other device or combination of devices that measure motion. More generally, any of a variety of sensors known in the art may be used to augment or supplement operation of the device 100 as contemplated herein.

The ultrasound probe may further include a sensor for illuminating the skin surface when the handheld ultrasound probe is placed for use against the skin surface. For example, the sensor may be a lighting source mechanically coupled to the handheld ultrasound probe and positioned to illuminate the skin surface during use of the ultrasound probe. The lighting source may be part of the sensor system of the ultrasound probe or the lighting source may be a separate device directed toward the ultrasound probe. Suitable lighting sources include an LED light or any other light capable of illuminating the skin surface during ultrasound scanning.

Another sensor that may be included in the device 100 is a camera 132. The camera 132 may be positioned to record a digital image of the target 136 during an ultrasound scan when the handheld ultrasound probe 112 is placed for use against the skin surface 137 of the target 136. The camera 132 also may be positioned to obtain a pose of the handheld ultrasound probe 112 as the ultrasound transducer 124 scans the target 136. The camera 132 may be mechanically coupled to the ultrasound transducer 124. In one aspect, the camera 132 may be rigidly mounted to the ultrasound transducer 124 and directed toward the skin surface 137 (when positioned for use) in order to capture images of the skin surface 137 and/or a target 134 adhered to the skin surface 137. In another aspect, the camera 132 may be mounted separate from the ultrasound probe 112 and directed toward an area of use of the ultrasound probe 112 so that the camera 132 can capture images of the ultrasound probe 112 in order to derive pose information directly from images of the ultrasound probe 112. Suitable cameras 132 may, for example, include any commercially available digital camera or digital video camera designed to capture images of sufficient quality for use as contemplated herein. The lighting source, sensor(s), and/or camera(s) may also include image intensification, magnification, active illumination, thermal imaging, or the like

The ultrasound probe 112 may have an integral structure with various components coupled directly to a body thereof, or one or more of the various functions of one or more of the components of the ultrasound probe may be distributed among one or more independent devices. For example, the camera, the lighting source, and any other sensors may be integrated into the ultrasound probe or they may be separate from the ultrasound probe, along with suitable communications and control systems where coordination of function is desired between the probe and the external components.

The ultrasound probe 112 may be used to capture an ultrasound image of a target 136 through a skin surface 137. A fiducial marker 134 with predetermined dimensions may be applied to the skin surface 137 of the target 136 that is to be scanned by the ultrasound probe 112. The fiducial marker 134 may have any desired dimension or shape such as a square, a rectangle, a circle and/or any other regular, irregular, and/or random shape and/or patterns. In an embodiment, the fiducial marker 134 may be a 3 mm×3 mm square. The fiducial marker 134 may be made of a thin material. Suitable materials include, but are not limited to, any materials that will not obstruct the transducer from obtaining an ultrasound scan of the target 136. The fiducial marker 134 may be adhered to the skin surface 137 of a target 136 using any suitable methods and/or any suitable adhesives. In another aspect, the fiducial marker 134 may be stamped, inked or otherwise applied to the skin surface using ink or any other suitable visually identifiable marking material(s).

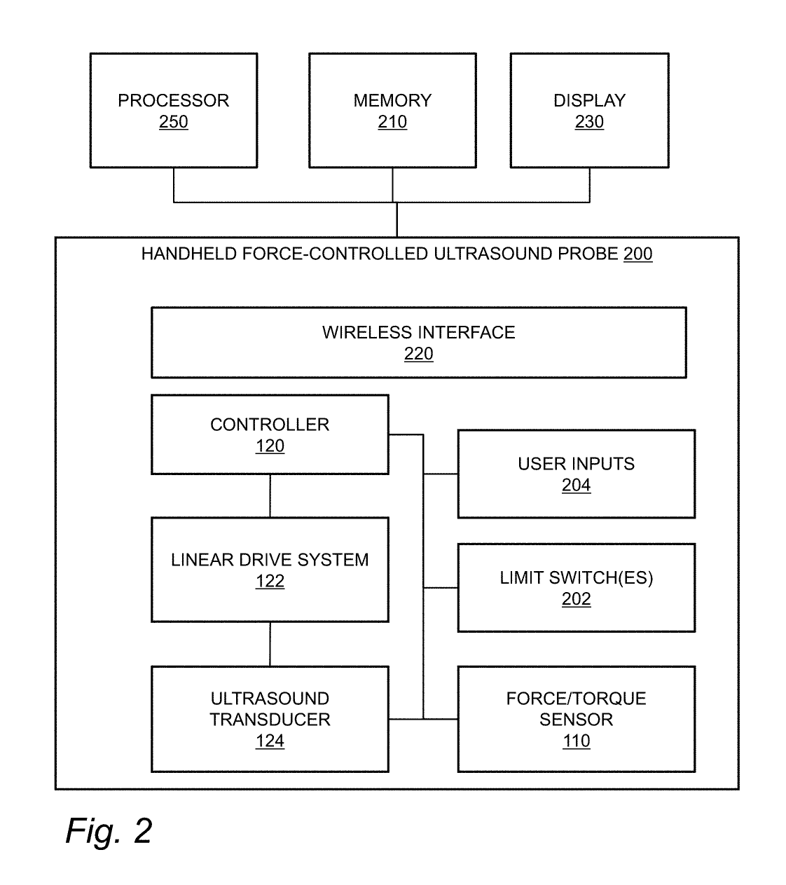

FIG. 2 is a schematic depiction of an ultrasound probe. The probe 200, which may be a force-controlled ultrasound probe, generally includes a sensor 110, a controller 120, a linear drive system 122, a position sensor 142, and an ultrasound transducer 124 as described herein.

In contrast to the probe 112 mounted in the device 100 as described in FIG. 1, the probe 200 of FIG. 2 may have the sensor 110, controller 120, and linear drive system 122 integrally mounted (as opposed to mounted in a separate device 100) in a single device to provide a probe 200 with an integral structure. In FIG. 2, the components are all operable to gather ultrasound images at measured and/or controlled forces and torques, as described with reference to FIG. 1. More generally, the various functions of the above-described components may be distributed across several independent devices in various ways (e.g., an ultrasound probe with integrated force/torque sensors but an external drive system, an ultrasound probe with an internal drive system but with an external control system, etc.). In one aspect, a wireless handheld probe 200 may be provided that transmits sensor data and/or ultrasound data wirelessly to a remote computer that captures data for subsequent analysis and display. All such permutations of the features described herein are within the scope of this disclosure.

The ultrasound transducer 124 can include a medical ultrasonic transducer, an industrial ultrasonic transducer, or the like. Like the ultrasound probe 112 described with reference to FIG. 1, it will be appreciated that a variety of embodiments of the ultrasound transducer 124 are possible, including embodiments directed to non-medical applications such as nondestructive ultrasonic testing of materials and objects and the like, or more generally, transducers or other transceivers or sensors for capturing data instead of or in addition to ultrasound data. Thus, although reference is made to an “ultrasound probe” in this document, the techniques described herein are more generally applicable to any context in which the transmission of energy (e.g., sonic energy, electromagnetic energy, thermal energy, etc.) from or through a target varies as a function of the contact force between the energy transmitter and the target.

Other inputs/sensors may be usefully included in the probe 200. For example, the probe 200 may include a limit switch 202 or multiple limit switches 202. These may be positioned at any suitable location(s) to detect limits of travel of the linear drive system 122, and may be used to prevent damage or other malfunction of the linear drive system 122 or other system components. The limit switch(es) may be electronically coupled to the controller 120 and provide a signal to the controller 120 to indicate when the limit switch 202 detects an end of travel of the linear drive system along the actuation axis. The limit switch 202 may include any suitable electro-mechanical sensor or combination of sensors such as a contact switch, proximity sensor, range sensor, magnetic coupling, and the like.

The position sensor 142 may be electronically and/or mechanically coupled to the limit switch(es) 202 to provide positioning information to the controller 120 concerning physical limits marked by the limit switch(es) 202. The position sensor 142 may provide position information to the controller 120 by tracking the travel of the probe 200 along the actuation axis 114 relative to the limit switch(es) 202. The controller 120 may receive the position information from the position sensor 142, determine the position of the probe 200, e.g., relative to the limit switch positions, and may provide control signals to movement of the probe 200 too close to the limit(s) of travel defined by the limit switch(es) 202.

The probe 200 may also or instead include one or more user inputs 204. These may be physically realized by buttons, switches, dials, or the like on the probe 200. The user inputs 204 may be usefully positioned in various locations on an exterior of the probe 200. For example, the user inputs 204 may be positioned where they are readily finger-accessible while gripping the probe 200 for a scan. In another aspect, the user inputs 204 may be positioned away from usual finger locations so that they are not accidentally activated while manipulating the probe 200 during a scan. The user inputs 204 may generally be electronically coupled to the controller 120, and may support or activate functions such as initiation of a scan, termination of a scan, selection of a current contact force as the target contact force, storage of a current contact force in memory for subsequent recall, or recall of a predetermined contact force from memory. Thus, a variety of functions may be usefully controlled by a user with the user inputs 204.

A memory 210 may be provided to store ultrasound data from the ultrasound transducer and/or sensor data acquired from any of the sensors during an ultrasound scan. The memory 210 may be integrally built into the probe 200 to operate as a standalone device, or the memory 210 may include remote storage, such as in a desktop computer, network-attached storage, or other device with suitable storage capacity. In one aspect, data may be wirelessly transmitted from the probe 200 to the memory 210 to permit wireless operation of the probe 200. The probe 200 may include any suitable wireless interface 220 to accommodate such wireless operation, such as for wireless communications with a remote storage device (which may include the memory 210). The probe 200 may also or instead include a wired communications interface for serial, parallel, or networked communication with external components. The memory 210 may be used to return the device to a previously recorded acquisition state.

A display 230 may be provided, which may receive wired or wireless data from the probe 200. The display 230 and memory 210 may be a display and memory of a desktop computer or the like, or may be standalone accessories to the probe 200, or may be integrated into a medical imaging device that includes the probe 200, memory 210, display 230, and any other suitable hardware, processor(s), and the like. The display 230 may display ultrasound images obtained from the probe 200 using known techniques. The display 230 may also or instead display a current contact force or instantaneous contact force measured by the sensor 110, which may be superimposed on a corresponding ultrasound image or in another display region of the display 230. Other useful information, such as a target contact force, an actuator displacement, or an operating mode, may also or instead be usefully rendered on the display 230 to assist a user in obtaining ultrasound images.

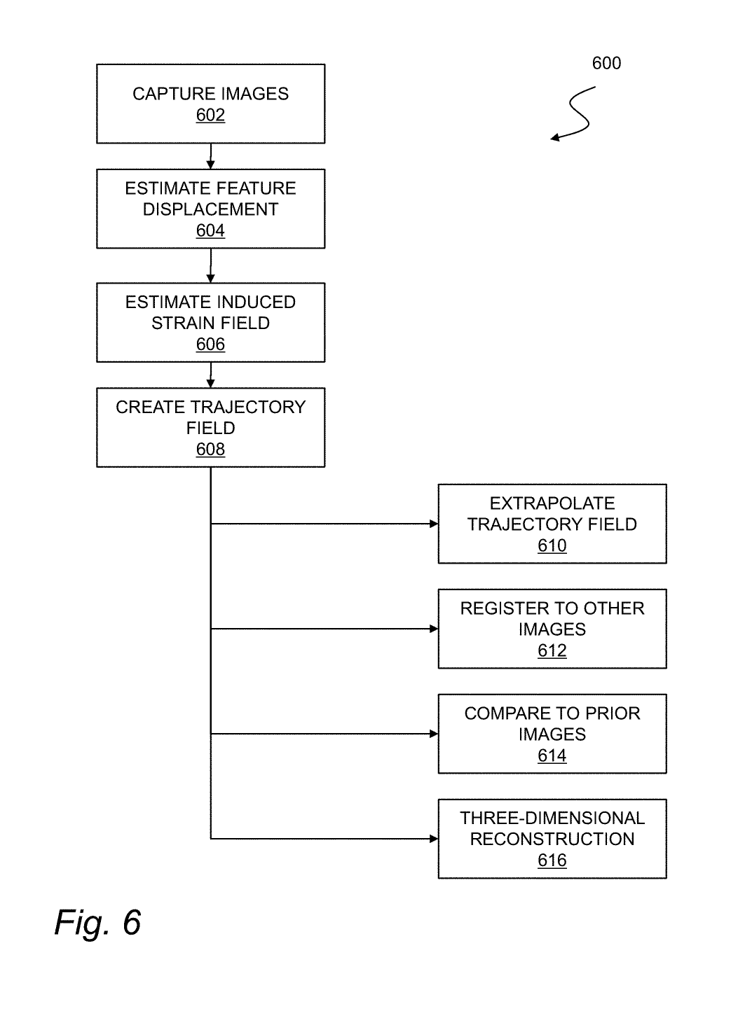

A processor 250 may also be provided. In one aspect, the processor 250, memory 210, and display 230 are a desktop or laptop computer. In another aspect, these components may be separate, or there may exist some combination of these. For example, the display 230 may be a supplemental display provided for use by a doctor or technician during an ultrasound scan. The memory 210 may be a network-attached storage device or the like that logs ultrasound images and other acquisition state data. The processor 250 may be a local or remote computer provided for post-scan or in-scan processing of data. In general, the processor 250 and/or a related computing device may have sufficient processing capability to perform the quantitative processing described herein. For example, the processor 250 may be configured to process an image of a subject from the ultrasound transducer 124 of the probe 200 to provide an estimated image of the subject at a predetermined contact force of the ultrasound transducer. This may, for example, be an estimate of the image at zero Newtons (no applied force), or an estimate of the image at some positive value (e.g., one Newton) selected to normalize a plurality of images from the ultrasound transducer 124. Details of this image processing are provided herein by way of example with reference to FIG. 6.

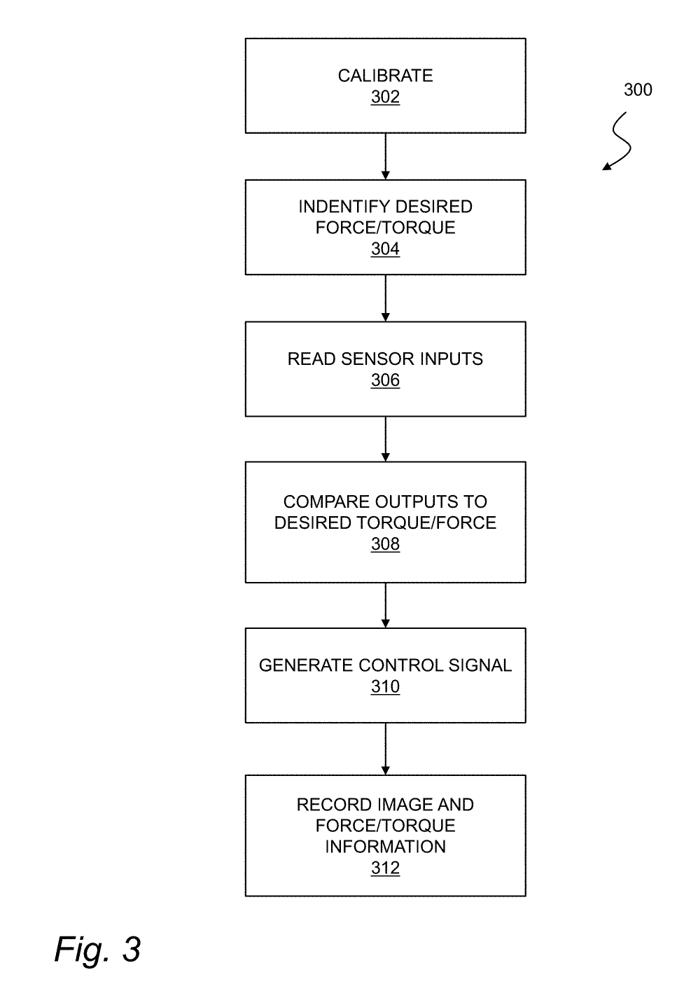

FIG. 3 is a flowchart of a process for force-controlled acquisition of ultrasound images. The process 300 can be performed, e.g., using a handheld ultrasound probe 112 mounted in a device 100, or a handheld ultrasound probe 200 with integrated force control hardware, or the like.

As shown in step 302, the process 300 may begin by calibrating the force and/or torque sensors. The calibration step is for minimizing (or ideally, eliminating) errors associated with the weight of the ultrasound probe or the angle at which the sensors are mounted with respect to the ultrasound transducer, and may be performed using a variety of calibration techniques known in the art.

To compensate for the mounting angle, the angle between the sensor axis and the actuation axis may be independently measured (e.g., when the sensor is installed). This angle may be subsequently stored for use by the controller to combine the measured forces and/or torques along each axis into a single vector, using standard coordinate geometry. For example, for a mounting angle θ, scaling the appropriate measured forces by sin(θ) and cos(θ) prior to combining them.

To compensate for the weight of the ultrasound probe, a baseline measurement may be taken, during a time at which the ultrasound probe is not in contact with the target. Any measured force may be modeled as due either to the weight of the ultrasound probe, or bias inherent in the sensors. In either case, the baseline measured force may be recorded, and may be subtracted from any subsequent force measurements. Where data concerning orientation of the probe is available, this compensation may also be scaled according to how much the weight is contributing to a contact force normal to the contact surface. Thus, for example, an image from a side (with the probe horizontal) may have no contribution to contact force from the weight of the probe, while an image from a top (with the probe vertical) may have the entire weight of the probe contributing to a normal contact force. This variable contribution may be estimated and used to adjust instantaneous contact force measurements obtained from the probe.

As shown in step 304, a predetermined desired force may be identified. In some implementations, the desired force is simply a constant force. For example, in imaging a human patient, a constant force of less than or equal 20 Newtons is often desirable for the comfort and safety of the patient.

In some implementations, the desired force may vary as a function of time. For example, it is often useful to “poke” a target in a controlled manner, and acquire images of the target as it deforms during or after the poke. The desired force may also or instead include a desired limit (minimum or maximum) to manually applied force. In some implementations, the desired force may involve a gradual increase of force given by a function F(t) to a force Fₘₐₓ at a time tₘₐₓ, and then a symmetric reduction of force until the force reaches zero. Such a function is often referred to as a “generalized tent map,” and may be given by the function G(t)=F(t) if t≤tₘₐₓ, and G(t)=Fₘₐₓ−F(t−tₘₐₓ) for t≥tₘₐₓ. When F is a linear function, the graph of G(t) resembles a tent, hence the name. In another aspect, a desired force function may involve increasing the applied force by some function F(t) for a specified time period until satisfactory imaging (or patient comfort) is achieved, and maintaining that force thereafter until completion of a scan. The above functions are given by way of example. In general, any predetermined force function can be used.

As shown in step 306, the output from the force and/or torque and position sensors may be read as sensor inputs to a controller or the like.

As shown in step 308, these sensor inputs may be compared to the desired force function to determine a force differential. In some implementations, the comparison can be accomplished by computing an absolute measure such as the difference of the sensor output with the corresponding desired sensor output. Similarly, a relative measure such as a ratio of output to the desired output can be computed. Additionally, output from the position sensor 142 may be compared to the positions of the limit switch(es) 202 to determine if the probe 200 is approaching an end of travel of the linear drive system 122. Further, many other functions can be used.

As shown in step 310, a control signal may be generated based on the comparison of actual-to-desired sensor outputs (or, from the perspective of a controller/processor, sensor inputs). The control signal may be such that the linear drive system is activated in such a way as to cause the measured force and/or torque to be brought closer to a desired force and/or torque at a given time. For example, if a difference between the measured force and the desired force is computed, then the drive system can translate the probe with a force whose magnitude is proportional to the difference, and in a direction to reduce or minimize the difference. Similarly, if a ratio of the desired force and measured force is computed, then the drive system can translate the probe with a force whose magnitude is proportional to one minus this ratio.

More generally, any known techniques from control theory can be used to drive the measured force towards the desired force. These techniques include linear control algorithms, proportional-integral-derivative (“PID”) control algorithms, fuzzy logic control algorithms, etc. By way of example, the control signal may be damped in a manner that avoids sharp movements of the probe against a patient's body. In another aspect, a closed-loop control system may be adapted to accommodate ordinary variations in a user's hand position. For example, a human hand typically has small positional variations with an oscillating frequency of about four Hertz to about twenty Hertz. As such, the controller may be configured to compensate for an oscillating hand movement of a user at a frequency between four Hertz and thirty Hertz or any other suitable range. Thus, the system may usefully provide a time resolution finer than twenty Hertz or thirty Hertz, accompanied by an actuation range within the time resolution larger than typical positional variations associated with jitter or tremors in an operator's hand.

As shown in step 312, the ultrasound probe can acquire an image, a fraction of an image, or more than one image. It will be understood that this may generally occur in parallel with the force control steps described herein, and images may be captured at any suitable increment independent of the time step or time resolution used to provide force control. The image(s) (or fractions thereof) may be stored together with contact force and/or torque information (e.g., instantaneous contact force and torque) applicable during the image acquisition. In some implementations, the contact force and/or torque information includes all the information produced by the force and/or torque sensors, such as the moment-by-moment output of the sensors over the time period during which the image was acquired. In some implementations, other derived quantities can be computed and stored, such as the average or mean contact force and/or torque, the maximum or minimum contact force and/or torque, and the like.

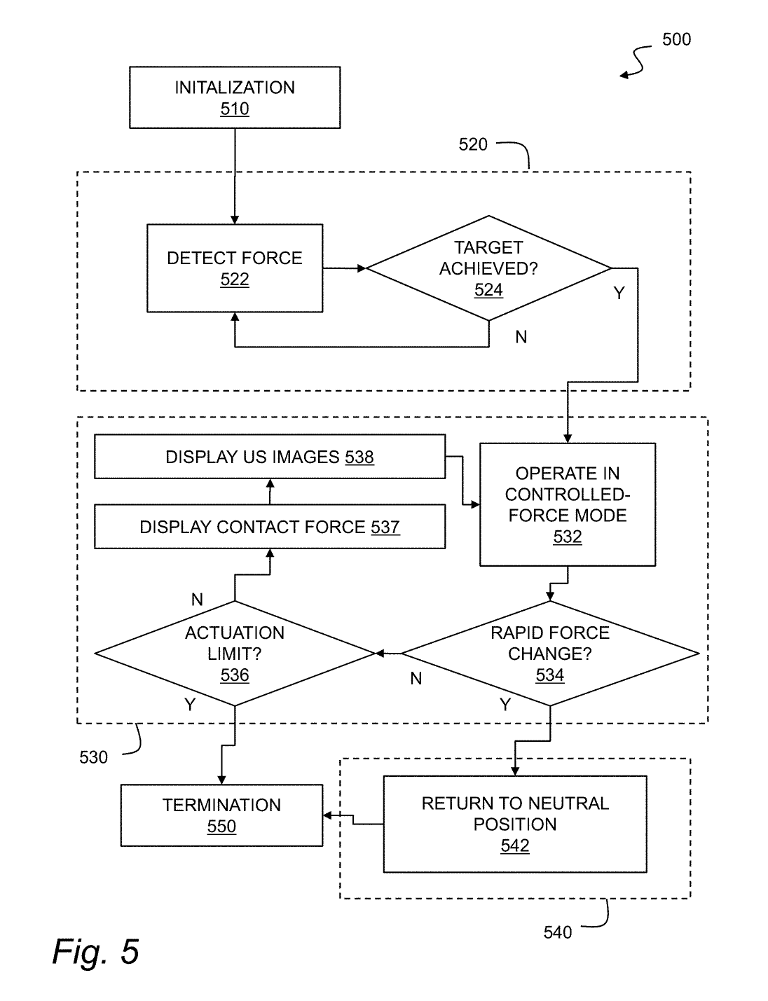

It will be understood that the steps of the methods described herein may be varied in sequence, repeated, modified, or deleted, or additional steps may be added, all without departing from the scope of this disclosure. By way of example, the step of identifying a desired force may be performed a single time where a constant force is required, or continuously where a time-varying applied force is desired. Similarly, measuring contact force may include measuring instantaneous contact force or averaging a contact force over a sequence of measurements during which an ultrasound image is captured. In addition, operation of the probe in clinical settings may include various modes of operation each having different control constraints. Some of these modes are described herein with reference to FIG. 5. Thus, the details of the foregoing will be understood as non-limiting examples of the systems and methods of this disclosure.

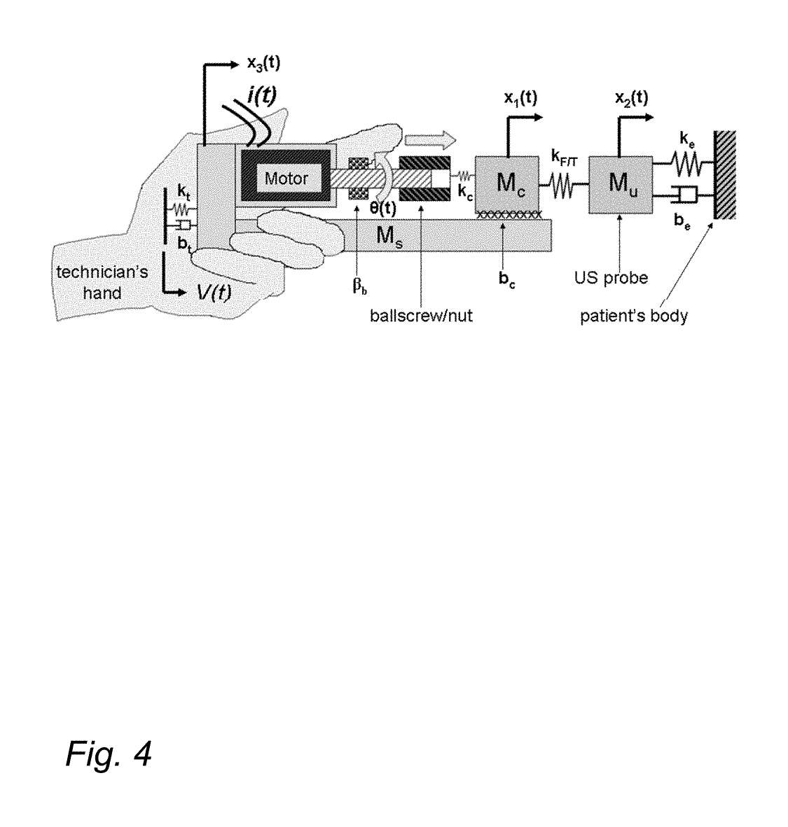

FIG. 4 shows a lumped parameter model of the mechanical system of a probe as described herein. While a detailed mathematical derivation is not provided, and the lumped model necessarily abstracts away some characteristics of an ultrasound probe, the model of FIG. 4 provides a useful analytical framework for creating a control system that can be realized using the controller and other components described herein to achieve force-controlled acquisition of ultrasound images.

In general, the model 400 characterizes a number of lumped parameters of a controlled-force probe. The physical parameters for an exemplary embodiment are as follows. Mᵤ is the mass of ultrasound probe and mounting hardware, which may be about 147 grams. M_{c} is the mass of a frame that secures the probe, which may be about 150 grams. Mₛ is the mass of the linear drive system, which may be about 335 grams. k_{F/T} is the linear stiffness of a force sensor, which may be about 1.1*10⁵ N/m. kₑ is the target skin stiffness, which may be about 845 N/m. bₑ is the viscous damping coefficient of the target, which may be about 1500 Ns/m. kₜ is the user's total limb stiffness, which may be about 1000 N/m. bₜ is the user's total limb viscous damping coefficient, which may be about 5000 Ns/m. b_{c} is the frame viscous damping coefficient, which may be about 0 Ns/m. k_{C} is the stiffness of the linear drive system, which may be about 3*10⁷ for an exemplary ballscrew and nut drive. K_{T} is the motor torque constant, which may be about 0.0243 Nm/A. β_{b} is be the linear drive system viscous damping, which may be about 2*10⁻⁴ for an exemplary ballscrew and motor rotor. L is the linear drive system lead, which may be about 3*10⁻⁴ for an exemplary ballscrew. Jₜₒₜ is the moment of inertia, which may be about 1.24*10⁻⁶ kgm² for an exemplary ballscrew and motor rotor.

Using these values, the mechanical system can be mathematically modeled, and a suitable control relationship for implementation on the controller can be determined that permits application of a controlled force to the target surface by the probe. Stated differently, the model may be employed to relate displacement of the linear drive system to applied force in a manner that permits control of the linear drive system to achieve an application of a controlled force to the target surface. It will be readily appreciated that the lumped model described herein is provided by way of illustration and not limitation. Variations may be made to the lumped model and the individual parameters of the model, either for the probe described herein or for probes having different configurations and characteristics, and any such model may be usefully employed provided it yields a control model suitable for implementation on a controller as described herein.

FIG. 5 is a flowchart depicting operating modes of a force-controlled ultrasound probe. While the probe described herein may be usefully operated in a controlled-force mode as discussed herein, use of the handheld probe in clinical settings may benefit from a variety of additional operating modes for varying circumstances such as initial contact with a target surface or termination of a scan. Several useful modes are now described in greater detail.

In general, the process 500 includes an initialization mode 510, a scan initiation mode 520, a controlled-force mode 530, and a scan termination mode 540, ending in termination 550 of the process 500.

As shown in step 510, an initialization may be performed on a probe. This may include, for example, powering on various components of the probe, establishing a connection with remote components such as a display, a memory, and the like, performing any suitable diagnostic checks on components of the probe, and moving a linear drive system to a neutral or ready position, which may for example be at a mid-point of a range of movement along an actuation axis.

As shown in step 522, the scan initiation mode 520 may begin by detecting a force against the probe using a sensor, such as any of the sensors described herein. In general, prior to contact with a target surface such as a patient, the sensed force may be at or near zero. In this state, it would be undesirable for the linear drive system to move to a limit of actuation in an effort to achieve a target controlled force. As such, the linear drive system may remain inactive and in a neutral or ready position during this step.

As shown in step 524, the controller may check to determine whether the force detected in step 522 is at or near a predetermined contact force such as the target contact force for a scan. If the detected force is not yet at (or sufficiently close to) the target contact force, then the initiation mode 520 may return to step 522 where an additional force measurement is acquired. If the force detected in step 522 is at or near the predetermined contact force, the process 500 may proceed to the controlled-force mode 530. Thus, a controller disclosed herein may provide an initiation mode in which a linear drive system is placed in a neutral position and a force sensor is measured to monitor an instantaneous contact force, the controller transitioning to controlled-force operation when the instantaneous contact force meets a predetermined threshold. The predetermined threshold may be the predetermined contact force that serves as the target contact force for controlled-force operation, or the predetermined threshold may be some other limit such as a value sufficiently close to the target contact force so that the target contact force can likely be readily achieved through actuation of the linear drive system. The predetermined threshold may also or instead be predictively determined, such as by measuring a change in the measured contact force and extrapolating (linearly or otherwise) to estimate when the instantaneous contact force will equal the target contact force.

As shown in step 532, the controlled-force mode 530 may begin by initiating controlled-force operation, during which a control system may be executed in the controller to maintain a desired contact force between the probe and a target, all as generally discussed herein.

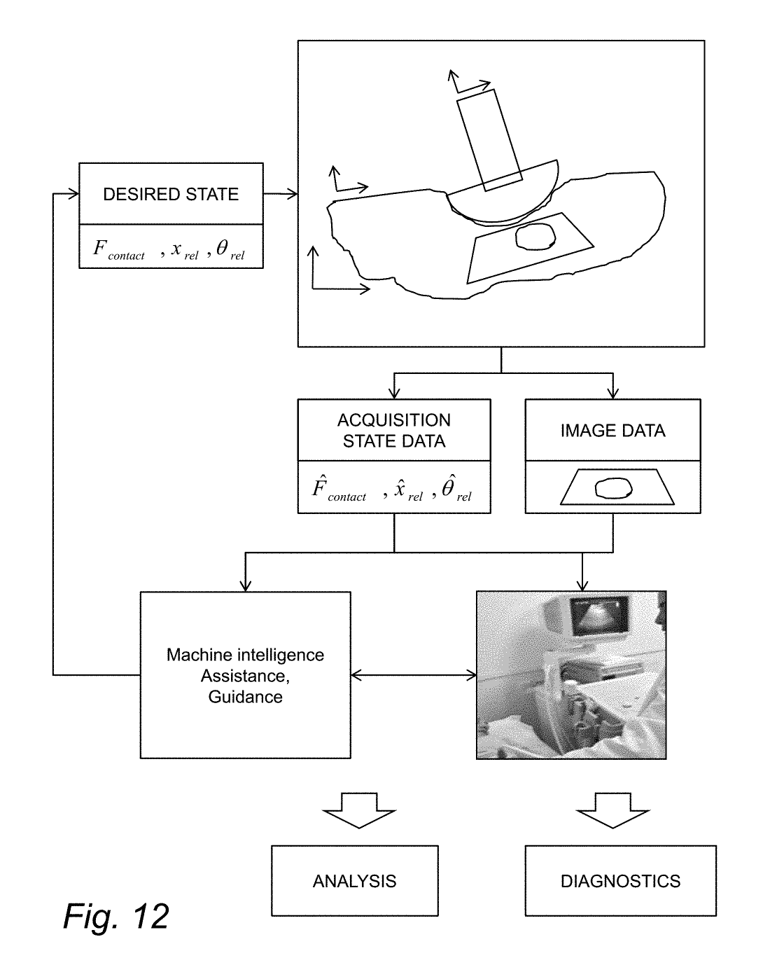

While in the controlled-force mode 530, other operations may be periodically performed. For example, as shown in step 534, the current contact force may be monitored for rapid changes. In general, a rapid decrease in contact force may be used to infer that a probe operator has terminated a scan by withdrawing the probe from contact with a target surface. This may be, for example, a step decrease in measured force to zero, or any other pattern of measured force that deviates significantly from expected values during an ongoing ultrasound scan. If there is a rapid change in force, then the process 500 may proceed to the termination mode 540. It will be appreciated that this transition may be terminated where the force quickly returns to expected values, and the process may continue in the controlled-force mode 530 even where there are substantial momentary variations in measured force. As shown in step 536, limit detectors for a linear drive system may be periodically (or continuously) monitored to determine whether an actuation limit of the linear drive system has been reached. If no such limit has been reached, the process 500 may continue in the controlled-force mode 530 by proceeding for example to step 537. In one example, if an actuation limit has been reached, then the process may proceed to termination 550 where the linear drive system is disabled. In another example, if an actuation limit has been reached, an endpoint avoidance strategy may be enabled to maintain the current position and force as described in more detail in FIG. 12. It will be appreciated that the process 500 may instead proceed to the termination mode 540 to return the linear drive system to a neutral position for future scanning.

As shown in step 537, a contact force, such as a force measured with any of the force sensors described herein, may be displayed on a monitor or the like. It will be appreciated that the contact force may be an instantaneous contact force or an average contact force for a series of measurements over any suitable time interval. The contact force may, for example, be displayed alongside a target contact force or other data. As shown in step 538, ultrasound images may be displayed using any known technique, which display may be alongside or superimposed with the force data and other data described herein.

As shown in step 542, when a rapid force change or other implicit or explicit scan termination signal is received, the process 500 may enter a scan termination mode 540 in which the linear drive system returns to a neutral or ready position using any suitable control algorithm, such as a controlled-velocity algorithm that returns to a neutral position (such as a mid-point of an actuation range) at a constant, predetermined velocity. When the linear drive system has returned to the ready position, the process 500 may proceed to termination as shown in step 550, where operation of the linear drive system is disabled or otherwise terminated.

Thus, it will be appreciated that a method or system disclosed herein may include operation in at least three distinct modes to accommodate intuitive user operation during initiation of a scan, controlled-force scanning, and controlled-velocity exit from a scanning mode. Variations to each mode will be readily envisioned by one of ordinary skill in the art and will fall within the scope of this disclosure. Thus, for example any one of the modes may be entered or exited by explicit user input. In addition, the method may accommodate various modes of operation using the sensors and other hardware described herein. For example, the controlled-force mode 530 may provide for user selection or input of a target force for controlled operation using, e.g., any of the user inputs described herein.

More generally, the steps described herein may be modified, reordered, or supplemented in a variety of ways. By way of example, the controlled-force mode of operation may include a controlled-velocity component that limits a rate of change in position of the linear drive system. Similarly, the controlled-velocity mode for scan termination may include a controlled-force component that checks for possible recovery of controlled-force operation while returning the linear drive system to a neutral position. All such variations, and any other variations that would be apparent to one of ordinary skill in the art, are intended to fall within the scope of this disclosure.

In general, the systems described herein facilitate ultrasound scanning with a controlled and repeatable contact force. The system may also provide a real time measurement of the applied force when each ultrasound image is captured, thus permitting a variety of quantitative analysis and processing steps that can normalize images, estimate tissue elasticity, provide feedback to recover a previous scan state, and the like. Some of these techniques are now described in greater detail.

FIG. 6 shows a process 600 for ultrasound image processing.

As shown in step 602, the process may begin with capturing a plurality of ultrasound images of an object such as human tissue. In general, each ultrasound image may contain radio frequency echo data from the object, and may be accompanied by a contact force measured between an ultrasound transducer used to obtain the plurality of ultrasound images and a surface of the object. The contact force may be obtained using, e.g., any of the hand-held, controlled force ultrasound scanners described herein or any other device capable of capturing a contact force during an ultrasound scan. The contact force may be manually applied, or may be dynamically controlled to remain substantially at a predetermined value. It will be appreciated that the radio frequency echo data may be, for example, A-mode or B-mode ultrasound data, or any other type of data available from an ultrasound probe and suitable for imaging. More generally, the techniques described herein may be combined with any force-dependent imaging technique (and/or contact-force-dependent imaging subject) to facilitate quantitative analysis of resulting data.

As shown in step 604, the process 600 may include estimating a displacement of one or more features between two or more of the ultrasound images to provide a displacement estimation. A variety of techniques are available for estimating pixel displacements in two-dimensional ultrasound images, such as B-mode block-matching, phase-based estimation, RF speckle tracking, incompressibility-based analysis, and optical flow. In one aspect, two-dimensional displacement estimation may be based on an iterative one-dimensional displacement estimation scheme, with lateral displacement estimation performed at locations found in a corresponding axial estimation. As described, for example, in U.S. Provisional Application No. 61/429,308 filed on Jan. 3, 2011 and incorporated herein by reference in its entirety, coarse-to-fine template-matching may be performed axially, with normalized correlation coefficients used as a similarity measure subsample estimation accuracy may be achieved with curve fitting. Regardless of how estimated, this step generally results in a two-dimensional characterization (e.g., at a feature or pixel level) of how an image deforms from measurement to measurement.

It will be understood that feature tracking for purposes of displacement estimation may be usefully performed on a variety of different representations of ultrasound data. Brightness mode (or “B-mode”) ultrasound images provide a useful visual representation of a transverse plane of imaged tissue, and may be used to provide the features for which displacement in response to a known contact force is tracked. Similarly, elastography images (such as stiffness or strain images) characterize such changes well, and may provide two-dimensional images for feature tracking.

As shown in step 606, the process 600 may include estimating an induced strain field from the displacement. In general, hyperelastic models for mechanical behavior work well with subject matter such as human tissue that exhibits significant nonlinear compression. A variety of such models are known for characterizing induced strain fields. One such model that has been usefully employed with tissue phantoms is a second-order polynomial model described by the strain energy function:

$$U = {\sum }_{i + j = 1}^{2} {{C}_{ij} ( {I}_{1} - 3 )}^{i} {( {I}_{2} - 3 )}^{j} + {\sum }_{i = 1}^{2} \frac{1}{{D}_{i}} {( {J}_{el} - 1 )}^{2 i} \qquad [ Eq . 1 ]$$

where U is the strain energy per unit volume, I₁ and I₂ are the first and second deviatoric strain invariant, respectively, and J_{eI} is the elastic volume strain. The variables Cᵢⱼ are the material parameters with the units of force per unit area, and the variables Dᵢ are compressibility coefficients that are set to zero for incompressible materials. Other models are known in the art, and may be usefully adapted to estimation of a strain field for target tissue as contemplated herein.

As shown in step 608, the process 600 may include creating a trajectory field that characterizes a displacement of the one or more features according to variations in the contact force. This may include characterizing the relationship between displacement and contact force for the observed data using least-square curve fitting with polynomial curves of the form:

x_{i,j}(f)=Σₖ₌₀^{N}α_{i,j,k}·f^{k} [Eq. 2]

y_{i,j}(f)=Σₖ₌₀^{N}β_{i,j,k}·f^{k} [Eq. 3]

where x_{i,j} and y_{i,j} pre the lateral and axial coordinates, respectively of a pixel located at the position (i,j) of a reference image, and α and β are the parameter sets determined in a curve fitting procedure. The contact force is f, and N denotes the order of the polynomial curves. Other error-minimization techniques and the like are known for characterizing such relationships, many of which may be suitably adapted to the creation of a trajectory field as contemplated herein.

With a trajectory field established for a subject, a variety of useful real-time or post-processing steps may be performed, including without limitation image correction or normalization, analysis of tissue changes over time, registration to data from other imaging modalities, feedback and guidance to an operator/technician (e.g., to help obtain a standard image), and three-dimensional image reconstruction. Without limiting the range of post-processing techniques that might be usefully employed, several examples are now discussed in greater detail.

As shown in step 610, post-processing may include extrapolating the trajectory field to estimate a location of the one or more features at a predetermined contact force, such as to obtain a corrected image. The predetermined contact force may, for example, be an absence of applied force (i.e., zero Newtons), or some standardized force selected for normalization of multiple images (e.g., one Newton), or any other contact force for which a corrected image is desired, either for comparison to other images or examination of deformation behavior. With the relationship between contact force and displacement provided from step 608, location-by-location (e.g., feature-by-feature or pixel-by-pixel) displacement may be determined for an arbitrary contact force using Eqs. 2 and 3 above, although it will be appreciated that the useful range for accurate predictions may be affected by the range of contact forces under which actual observations were made.

As shown in step 612, post-processing may include registering an undistorted image to an image of an object obtained using a different imaging modality. Thus, ultrasound results may be registered to images from, e.g., x-ray imaging, x-ray computed tomography, magnetic resonance imaging (“MRI”), optical coherence tomography, positron emission tomography, and the like. In this manner, elastography data that characterizes compressibility of tissue may be registered to other medical information such as images of bone and other tissue structures.

As shown in step 614, post-processing may include comparing an undistorted image to a previous undistorted image of an object. This may be useful, for example, to identify changes in tissue shape, size, elasticity, and composition over a period of time between image captures. By normalizing a contact force or otherwise generating corrected or undistorted images, a direct comparison can be made from one undistorted image to another undistorted image captured weeks, months, or years later.

As shown in step 616, post-processing may also or instead include capturing multiple undistorted images of a number of transverse planes of an object such as human tissue. Where these images are normalized to a common contact force, they may be registered or otherwise combined with one another to obtain a three-dimensional image of the object. The resulting three-dimensional image(s) may be further processed, either manually or automatically (or some combination of these), for spatial analysis such as measuring a volume of a specific tissue within the object, or measuring a shape of the tissue.

Still more generally, any post-processing for improved imaging, diagnosis, or other analysis may be usefully performed based on the quantitative characterizations of elastography described herein. These and related techniques are referred to generally herein as quantitative elastography, although it will be understood that this phrase is used for convenience and not by way of limitation. For example, an ultrasound image of an artery may be obtained, and by measuring an amount of compression in the artery in response to varying contact forces, blood pressure may be estimated. Similarly, by permitting reliable comparisons of time-spaced data, better diagnosis/detection of cancerous tissue can be achieved. Any such ultrasound imaging applications that can be improved with normalized data can benefit from the inventive concepts disclosed herein.

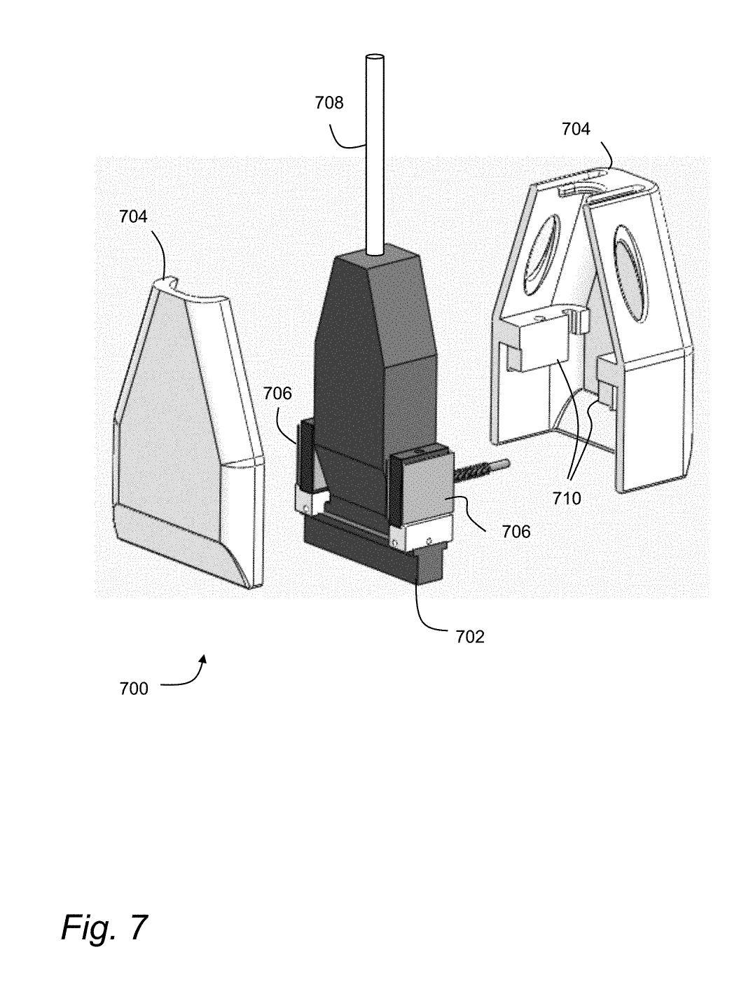

FIG. 7 shows an exploded view of an ultrasound probe with a distributed load sensor. In general, the device 700 may include an ultrasound transducer 702, an enclosure 704 (shown in two parts), and two or more force sensors 706.

The ultrasound transducer 702 may include any ultrasound transducer system suitable for imaging as contemplated herein. This may, for example, be any of a variety of commercially available or other ultrasound imaging devices, and may generally include an acoustic stack that integrates components known in the art such as a lens, matching layer, piezoelectric crystal elements and so forth, along with cables 708 or other electromechanical linkages to couple the functional elements of the ultrasound transducer 702 to a controller, processor, or other control system or the like. Clinical probes typically employ a one hundred twenty-eight element array of piezoelectric transducers, but the ultrasound transducer 702 may more generally include a transducer array with any number of ultrasound transducer elements (e.g., two or more discrete ultrasound transducers) suitable for imaging as described herein.

The enclosure 704 may generally provide a handle for the ultrasound transducer 702 to permit manipulation of the ultrasound transducer 702 by a user. The enclosure 704 is illustrated in FIG. 7 as a two-part exterior housing suitable for retrofit assembly onto a commercially available ultrasound system or component. However, the enclosure 704 may more generally be formed of any material or combination of materials, and in any shape suitable for gripping and manipulating the ultrasound transducer 702 while capturing ultrasound images. In one aspect, the multi-part enclosure 704 may usefully be configured so that the force sensors 706 all couple directly to a single physical component, e.g., at fixtures 710 configured to securely mechanically mate with the force sensors 706. This approach may advantageously mitigate artifacts associated with independent movement of the force sensors 706 during use.

The force sensors 706 are coupled between the enclosure 704 and the ultrasound transducer 702 to permit a measurement of an instantaneous contact force applied to the ultrasound transducer 702 (through the enclosure 704) by a target surface. In general, the force sensors 706 may be positioned to measure an instantaneous contact force at two or more corresponding locations around the ultrasound transducer 702 when the ultrasound transducer is placed against a surface of an object for use in order to facilitate the measurement of a spatially distributed contact force between the two, referred to herein as a force distribution. It will be understood that, for the sake of clear and efficient explanation of the system and related aspects of operation of the system, reference is generally made herein to a force distribution. However, unless otherwise specified or made clear from the context, any reference to force herein should be understood to be inclusive of any suitable measure of, or proxy for, a mechanical load. For example, the system may measure a pressure distribution, a load distribution, or the like. Thus, references herein to a force sensor, contact force, or distributed force should be understood to include a pressure sensor, surface pressure, and distributed pressure respectively, and systems measuring or applying pressures are intended to fall within the scope of this disclosure as though fully set out in all references to force herein, unless otherwise stated specifically to the contrary.

As a user grips the enclosure 704 and places the ultrasound transducer 702 in contact with the target surface, the instantaneous contact force between the target surface and the ultrasound transducer is measured and communicated, e.g., through the cables 708 to a processor along with other information in order to facilitate force sensing and force-based image processing. In general, the force sensors 706 may be any component(s) suitable for detecting force and providing corresponding signals to a processing system. For example, this may include strain gauges, force sensing resistors, commercially available load cells and the like. In one embodiment, the force sensors 706 may be LSB200 load cells commercially available from Futek Advanced Sensor Technology, Inc.

By using multiple force sensors 706, e.g., along a transducer array and/or within the transverse imaging plane of the ultrasound transducer 702, many useful calculations and adjustments can be made. In one aspect, the multiple force measurements can be used to determine a force distribution along a contact region between the ultrasound transducer 702 and a target surface. In one aspect, this can improve ultrasound images by permitting adjustments to, e.g., shear wave elastography measurements, quantitative elastography calculations, and image normalization. For example, an image can be normalized, using quantitative elastography, to estimate and display a transverse plane image at a fixed and/or uniformly distributed contact force, thus potentially reducing image variability in real time as the position and applied force from a human user varies during a scan. In another aspect, the force distribution may be used to estimate a pose of the ultrasound transducer 702 relative to a target surface, and/or to provide feedback to a user on proper positioning and pressure for a scan.

In general, the two force sensors 706 may be positioned within a transverse imaging plane of the ultrasound transducer 702 or otherwise positioned to measure a force distribution within that imaging plane. While two force sensors 706 are shown, it will be understood that three or more force sensors 706 may be used. For example, the system may include a third force sensor 706 coupled between the enclosure 704 and the ultrasound transducer 702 and positioned to measure a third instantaneous contact force at a third corresponding location around the ultrasound transducer 702 when the ultrasound transducer is placed against the surface of the object for use. This third force sensor 706 may, e.g., lie along a line between the two other force sensors 706, and may be used as a third measurement in order to improve accuracy of the force distribution calculated along the line. A force sensor 706 may also or instead be positioned away from the line between the two other force sensors 706 in order to provide additional measurements off of an axis of the line, e.g., to provide an additional dimension for force distribution measurements.

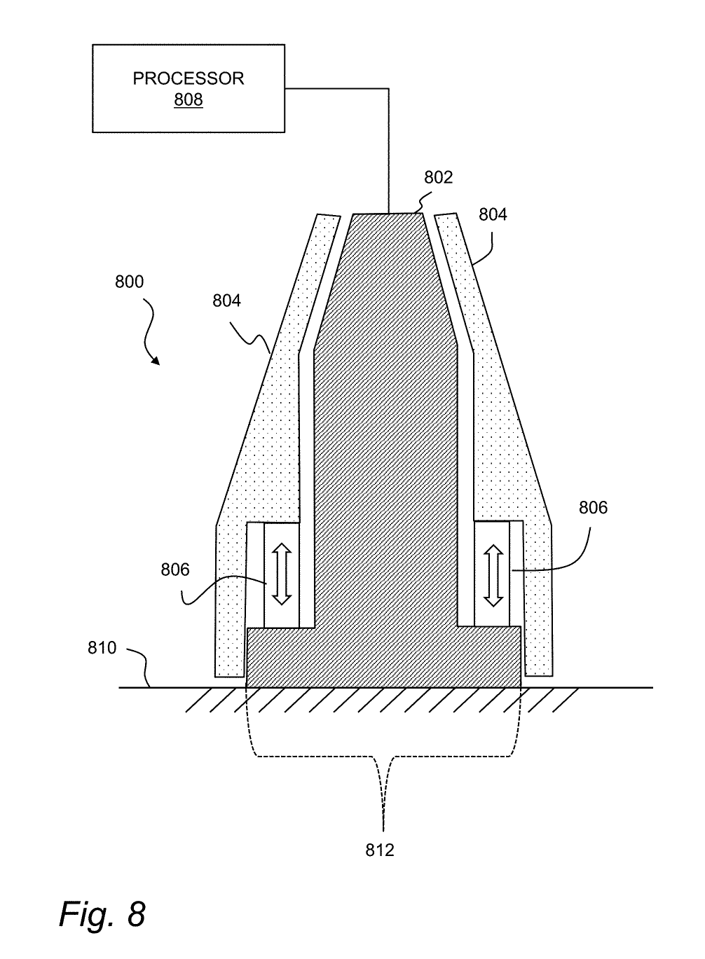

FIG. 8 shows a cross section of an ultrasound probe with a distributed load sensor. The ultrasound probe 800, which may be any of the ultrasound probes described herein, may include an ultrasound transducer 802, an enclosure 804 and two or more force sensors 806 such as any of those described above. The ultrasound probe 800 may also include a processor 808 coupled in a communicating relationship with the force sensors 806 and ultrasound transducer 802, e.g., directly or through other components such as data acquisition hardware, ultrasound imaging hardware and so forth. These additional components are well known in the art, and for simplicity they are not described in detail here.

When placed in contact with a target surface 810, each force sensor 806 measures an instantaneous contact force at a corresponding location. In general, the applied force of the target surface 810 is split among the force sensors 806, and a force distribution within the contact region 812—generally but not necessarily coextensive with a locus of physical contact between the target surface 810 a face of the ultrasound transducer 802—can be calculated or estimated by the processor 808, which may be configured to calculate a force distribution across the ultrasound transducer 802 based on a set of signals from the force sensors. The force distribution may be determined using, e.g., a beam model with a distributed load and two point forces (for a two-sensor system) or any other suitable technique for calculating a distributed load or the like.

The force distribution across the ultrasound transducer may be used in a variety of ways to improve the quality of images and/or normalize ultrasound images captured under different loadings. For example, shear wave elasticity is a known technique for mapping elasticity and stiffness of tissue with ultrasound imaging by applying an acoustic radiation force and measuring the speed of the resulting shear wave. In general, when force is applied to tissue, the elasticity of the tissue increases, which results in an increased shear wave speed in the compressed region(s). By measuring a force distribution of an ultrasound transducer across the surface of a region of interest, the resulting effects on shear wave speed can be estimated and the shear wave measurements or the resulting, calculated shear wave elasticity can be adjusted to reflect, e.g., a uniform force distribution. In another aspect, a user may be notified of the non-uniform force distribution e.g., with a visual or auditory alert and/or instructions to adjust a contact angle of the ultrasound transducer with a target surface.

In another aspect, the force distribution may be used to make spatial adjustments to ultrasound images. For example, a processor may be configured to create a two-dimensional image of a transverse plane of an object, e.g., based on signals from the ultrasound transducer and/or shear wave elasticity measurements. The processor may usefully apply quantitative elastography as described above, or any other suitable techniques, in order to normalize the two-dimensional image to a predetermined contact force, such as a contact force with a predetermined magnitude and/or distribution (e.g., uniform or substantially uniform), based upon the measured force distribution across the ultrasound transducer.

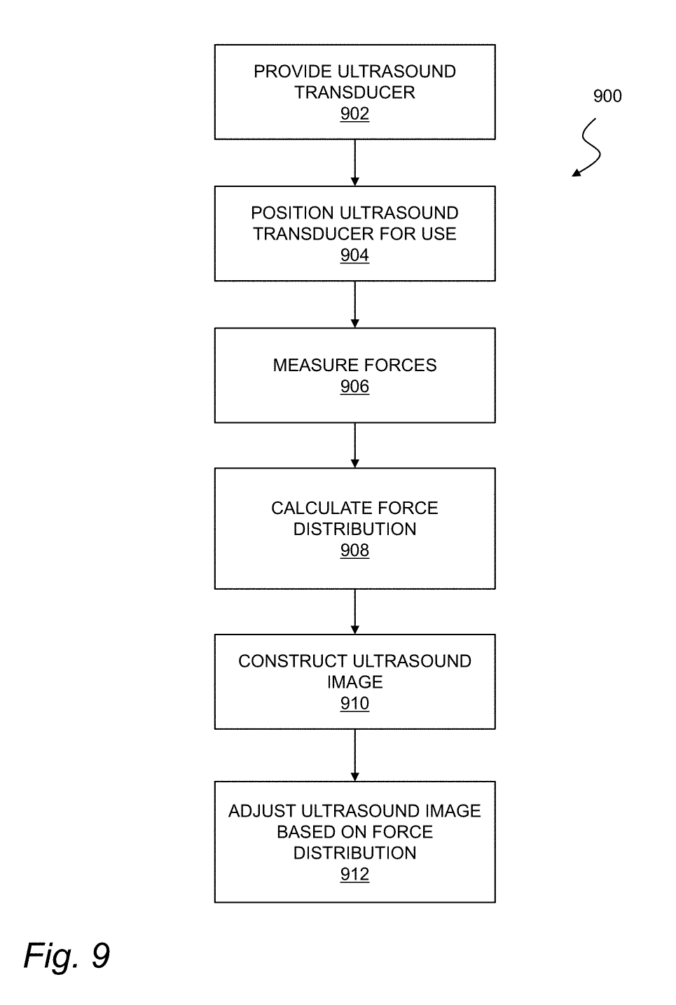

FIG. 9 shows a method for using an ultrasound transducer with distributed force sensing.

As shown in step 902, the method 900 may include providing an ultrasound transducer such as any of the ultrasound transducers described herein. This may, for example, include an ultrasound transducer coupled to an enclosure by two force sensors at two predetermined positions relative to the ultrasound transducer and the enclosure. The two force sensors may, for example, lie in a transverse plane for capturing images of the object with the ultrasound transducer. In another aspect, the ultrasound transducer may be coupled to the enclosure through three or more force sensors.

As shown in step 904, the method 900 may include positioning the ultrasound transducer for use. For example, this may include applying a positioning force to the enclosure to contact the ultrasound transducer to a target surface of an object, e.g., by manually gripping the enclosure and manipulating the ultrasound transducer into contact with the target surface.

As shown in step 906, the method 900 may include measuring forces from the ultrasound probe, such as a force at each of the two force sensors.

As shown in step 908, the method 900 may include calculating a force distribution based on the measurements from the force sensors. This may, for example, include calculating a force distribution over a contact region between the ultrasound transducer and the target surface based on the two predetermined positions and a signal received from each of the two force sensors. This may include calculating the total load, calculating the shear force or bending moment across the contact region, e.g., by modeling the contact surface as a simple beam, or otherwise modeling the distributed force across a face of the ultrasound transducer. In one aspect, a number of point forces may be interpolated, e.g., at locations spatially consistent with shear wave elasticity calculations, or otherwise convenient for supporting tissue elasticity estimates or image normalization.

As shown in step 910, the method 900 may include constructing an ultrasound image. This may, for example, include processing signals from the ultrasound transducer to construct an image in a transverse plane through the object, or any other suitable one-dimensional, two-dimensional or three-dimensional image, based on signals from the ultrasound transducer. image or other image from regions of the object below the target surface. This may also or instead include receiving ultrasound signals from the ultrasound transducer and calculating a shear wave elasticity within a transverse plane through the object based on the ultrasound signals.

As shown in step 912, the method 900 may include adjusting an ultrasound image based on the force distribution measured above. For example, this may include adjusting the shear wave elasticity based on the force distribution across the ultrasound transducer, e.g., to account for changes in elasticity (and shear wave speed) for tissue under compression due to the applied force. It will be understood that in this case, and more generally as contemplated herein, some processing steps may be performed by an existing ultrasound imaging system and some processing steps may be performed by a supplemented processing system added, e.g., to an existing commercial ultrasound product. Thus, while an integrated system may usefully be constructed using the principles described herein, many of these techniques may also be conveniently retrofitted to an existing commercial system. Thus, in one aspect, the ultrasound system may calculate shear wave elasticity in a region of interest within an object, and a supplemental processor or the like by used to adjust the shear wave elasticity based on a measured force distribution. In another aspect, the processor may associate particular ultrasound shear wave velocity measurements with a corresponding force or pressure distribution in any manner suitable for improved image processing or quantitative analysis.

In another aspect, the two-dimensional ultrasound image (or other ultrasound image) may be normalized to a predetermined contact force based on the force distribution across the ultrasound transducer. For example, in order to remove tissue distortion associated with the way in which individual users manipulate an ultrasound probe, the ultrasound images may be normalized to estimate, e.g., the image under a one Newton load uniformly distributed across the contact region. In one aspect, normalizing a two-dimensional image in the transverse plane to the predetermined contact force may include applying quantitative elastography, e.g., using the techniques described above, to normalize the two-dimensional image to an adjusted image representative of a uniform contact force across the contact region.

Similarly, there is disclosed herein a computer program product for processing ultrasound signals from an ultrasound transducer coupled to an enclosure through two force sensors at two predetermined positions. The computer program product may include computer executable code embodied in a non-transitory computer readable medium that, when executing on one or more computing devices (such as an ultrasound probe controller and/or ultrasound imaging system), performs the steps of: receiving a force signal from each of the two force sensors; calculating a force distribution over a contact region between the ultrasound transducer and a target surface of an object based on the two predetermined positions and a signal received from each of the two force sensors; and adjusting an ultrasound image of the object acquired with the ultrasound transducer based on the force distribution over the contact region.

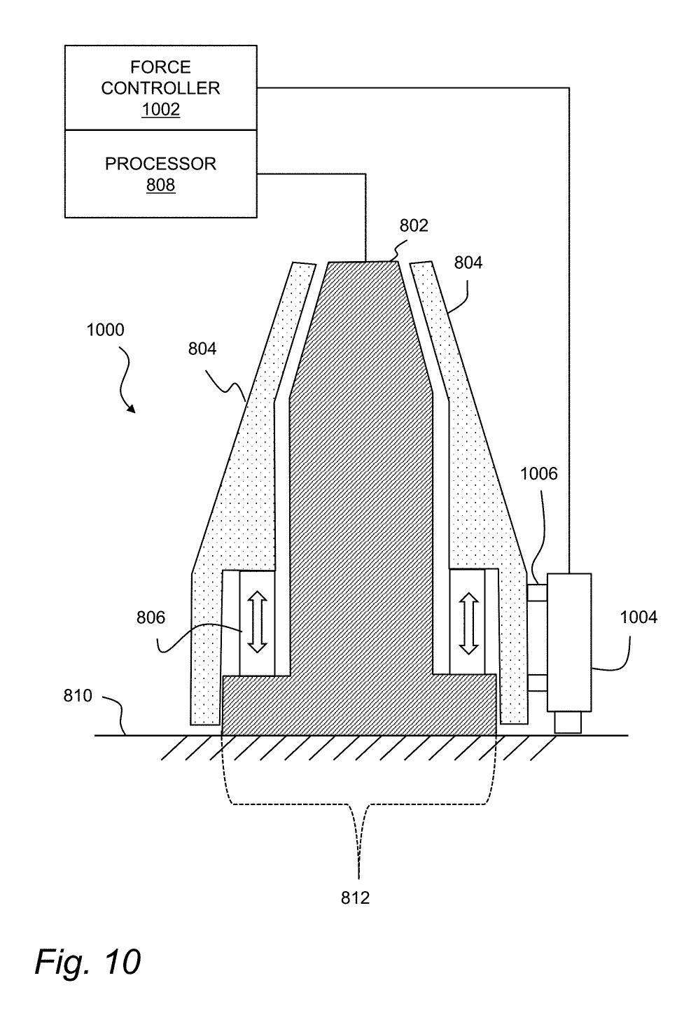

FIG. 10 shows a device for applying a force to an object. In general, the device 1000 may include any of the devices described herein such as force-sensing ultrasound probe or other ultrasound imaging device or the like. In general, the device 1000 may be configured, e.g., as described below, to deliver a predetermined force signature such as a series of taps or the like to a target surface 810 in order to create a detectable mechanical wave in an object. A force-sensing probe can then detect this wave with different hardware systems, e.g., an independent ultrasound transducer system and force sensing system, and use the detected wave to synchronize these systems for improved imaging.

The device 1000 may include a force controller 1002 configured to apply a predetermined force signature to a target surface 810 of an object in contact with an ultrasound transducer 802. In one aspect, the device 1000 may be a force-controlled ultrasound probe such as any of those described above, and the force controller 1002 may be the controller that controls an instantaneous contact force applied by the probe to a contact surface. In one aspect, the force controller 1002 may use preexisting hardware in a force-controlled ultrasound probe, and may include the linear actuator and related systems described above. Thus, while illustrated separately, the force controller 1002 may be a processor, microcontroller or the like that is used to control a force-controlled and/or force sensing ultrasound probe. In another aspect, a separate hardware system may be provided to support the delivery of the predetermined force signature. For example, this may include an actuator 1004 such as a linear motor, a pneumatic actuator, a piezoelectric motor, or other hardware capable of delivering a sequence of taps or other force signature to the target surface 810. The actuator 1004 may by coupled to the enclosure 804, e.g., with an isolator 1006 that isolates vibration and other mechanical motion of the actuator 1004 from the enclosure 804 and other connected components. The isolator may use any of a variety of passive and active isolation techniques in order to prevent or reduce mechanical crosstalk between the actuator 1004 and the other system components. In another aspect, the actuator 1004 may be mechanically independent, e.g., not physically coupled to the enclosure 804 in order to mitigate signal noise for the ultrasound transducer 802, force sensors 806 and the like. Thus, for example, the actuator 1004 may be a separate handheld or body mounted actuator for applying suitable mechanical pulses or the like at or near the region of interest.

It should be appreciated that the force controller 1002 does not generally need to be mechanically or electronically coupled to the enclosure 804 or other components of the device 1000. While the force controller 1002 may usefully create a distinguishable mechanical wave for detection by the force sensors 806 and the ultrasound transducer 802, these two hardware systems need to synchronize to each other, not the source of the predetermined force signature. Thus, it is generally more important that the predetermined force signature be clearly detectable within signals from the two hardware systems than that the source of the predetermined force signature be coupled to the other systems in any particular way. In one aspect, the predetermined force signature may be a manual tapping of the surface, e.g., with three sharp taps, which may create a characteristic wave suitable for synchronizing the force sensors 806 and ultrasound transducer 802. In another aspect, a machine-generated signal may advantageously be used to deliver a sequence of taps or other impulses or the like in order to ensure that the sequence of taps are spaced apart at consistent and predictable intervals, which may further improve detection and synchronization.

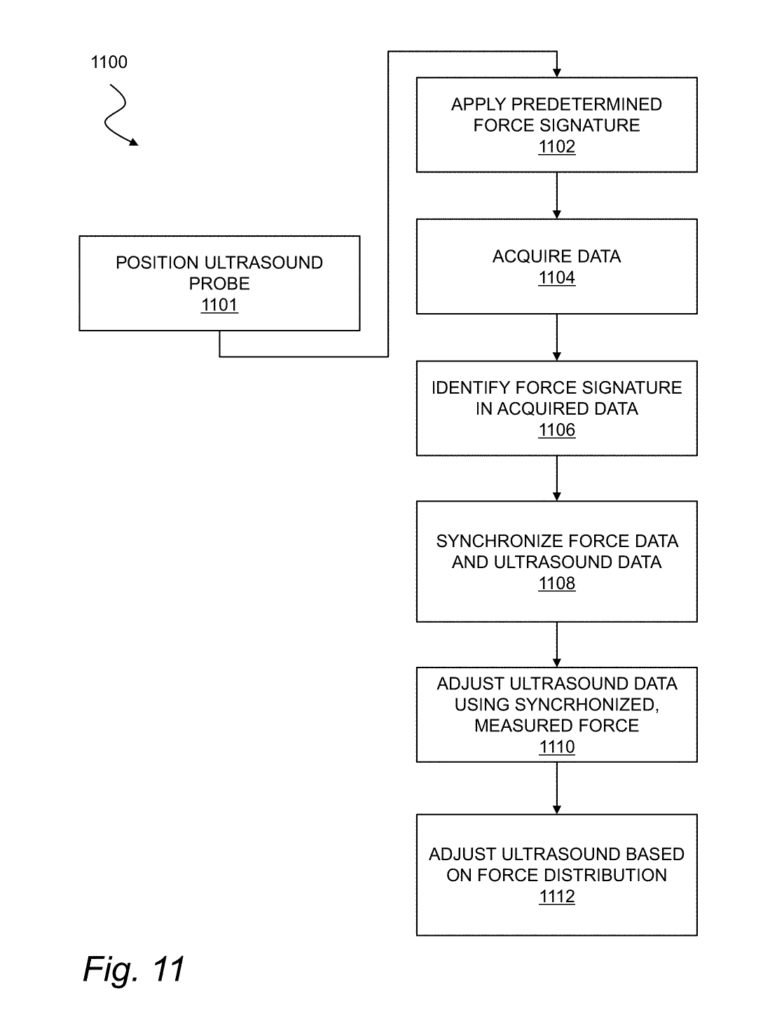

FIG. 11 shows a method for synchronizing force data and ultrasound data from a force-sensing ultrasound probe. In general, a distinctive force signature such as a sequence of discrete taps is applied to a surface of interest, and responsive signals from the force sensors and ultrasound transducer can be used to synchronize force data to ultrasound data for the probe.

As shown in step 1101, the method 1100 may begin with positioning an ultrasound probe for use, such as by contacting an ultrasound probe with an ultrasound transducer and a force sensor to a target surface of an object. The ultrasound probe may include any of the ultrasound transducers described herein, such as an ultrasound transducer array. As another example, the force sensor may include a multi-cell load sensor for measuring a distributed force across a contact surface of the ultrasound transducer, e.g. a two or more load cells positioned to measure a force distribution across the ultrasound transducer when placed for use on the target surface. More generally, this may include any of the other force sensors or the like described herein including force sensors for capturing single point measurements of instantaneous contact force, multiple point measurements of instantaneous contact force, or distributed measurements of contact force, as well as combinations of the foregoing.

As shown in step 1102, the method 1100 may include applying a predetermined force signature to a surface of an object. In one aspect, this may be manually performed, e.g., by manually applying a number of taps with constant or varying frequency and amplitude, or by more generally tapping, shaking, or otherwise mechanically stimulating the surface. In this context, it will be understood that a constant frequency or amplitude is intended to include any ordinary amount of variability that might be present in a manual operation performed by a human technician or the like. In another aspect, this may include automatically applying the predetermined force signature by using a device to electromechanically or otherwise apply a number of taps with constant or varying frequency and amplitude. This may, for example, include driving a force controller to apply a predetermined force signature to a surface of an object in contact with an ultrasound transducer of the force-sensing ultrasound probe. Applying a predetermined force signature may also or instead include applying a predetermined force signature with the ultrasound probe, e.g., by operating a linear actuator of a force-controlled probe to apply an instantaneous contact force having the predetermined force signature, or by operating a separate hardware system configured to apply the predetermined force signature under control of a processor or other controller for the ultrasound probe.

As discussed above, a variety of signatures may usefully be employed, and in general, any signature suitable for detection by both the force sensors and the ultrasound transducer may be used as a predetermined force signature as contemplated herein. In one aspect, the predetermined force signature includes two or more high frequency variations to an instantaneous contact force between the ultrasound transducer and the surface of the object. It has been observed that a single tap may not be sufficiently distinct for reliable detection within an otherwise noisy force or ultrasound data stream. At the same time, additional repetition, e.g., with four, five or more taps or the like, provides only marginally improved detection. Thus, in one aspect, the predetermined force signature may also or instead include three consecutive taps to the object, although more or fewer taps may also or instead be used.

As shown in step 1104, the method 1100 may include acquiring data from the force-sensing ultrasound probe, such as a first data stream from the ultrasound transducer and a second data stream from the force sensor. The first data stream may include ultrasound signals used to create an ultrasound image. The second data stream may be from a force sensor of the force-sensing ultrasound probe such as a load cell or any of the other force sensors described above. In general, while the acquisition of data specifically responsive to the force signature does not occur until the force signature is applied, a step of initiating an acquisition of a first data stream from the ultrasound transducer and a second data stream from the force sensor may occur at any time prior to (or possibly during) application of the force signature, and data may be continuously or intermittently acquired during operation of the ultrasound probe.

As shown in step 1106 the method 1100 may include identifying the force signature in the ultrasound and force data. For example, this may include locating a first moment within the first data stream and a second moment within the second data stream coincident with the predetermined force signature. For example, this may include locating a first moment within the first data stream by analyzing ultrasound images to identify a moment coincident with a physical deformation within the object resulting from the predetermined force signature. This may also or instead include locating a second moment within the second data stream coincident with a measured contact force resulting from the predetermined force signature. While each signal may be analyzed independently to identify artifacts of the predetermined force signature, the signals may also be analyzed together using any of a variety of suitable signal processing techniques. For example, identifying the force signature in the ultrasound and force data may include applying a matched filter to align force data from the second data stream with an integrated optical flow velocity within an ultrasound video image based on the first data stream. More generally, any signal processing technique, image processing technique or other processing techniques or the like suitable for detecting the force signature within the force data and the ultrasound data may usefully be employed to identify the force signature as contemplated herein.

More generally, while described as discrete steps of identifying the signature in two data streams and then synchronizing the data streams, it will be understood that a variety of signal processing techniques may be used to directly or indirectly identify the first and second moment without performing these steps independently. For example, a suitably configured frequency domain phase detector may be used to identify a time offset or phase between patterns in the force data and the ultrasound data, which may be used to align data in subsequent synchronization steps without explicitly identifying the time domain force signature in each data stream. These and any other suitable time domain or frequency domain signal processing techniques may be used in a synchronization process as contemplated herein, and the steps of identifying the force signature and synchronizing data streams as shown in FIG. 11 are intended to include all such techniques for synchronizing the data streams unless a more specific meaning is otherwise stated or clear from the context.

As shown in step 1108, the method 1100 may include synchronizing the first data stream (e.g., the ultrasound data or images/video reconstructed therefrom) with the second data stream (e.g., the force sensor data) using a time base that matches the first moment where the force signature appears within the first data stream to the second moment where the force signature appears within the second data stream. In one aspect, the time base may be a time base that matches the temporal origin and sampling rate of both signals to one another.

As shown in step 1110, the method 1100 may include adjusting the ultrasound data based on the (synchronized) force data. For example, this may include adjusting a subsequent ultrasound image from the ultrasound transducer according to a synchronized measurement of a contact force detected with the force sensor. More generally, this may include applying any of the quantitative elastography or other techniques described herein, e.g., to refine elasticity measurements, to normalize ultrasound images (e.g., to a predetermined contact force), or otherwise adjust images, calculations or the like derived from ultrasound data.

In another aspect, there is disclosed herein an ultrasound device that synchronizes independent force measurements and ultrasound measurements. The device may include an ultrasound transducer, an enclosure with a handle for the ultrasound transducer, a force sensor coupled between the enclosure and the ultrasound transducer and positioned to measure an instantaneous contact force between the ultrasound transducer and a target surface of an object, a force controller configured to apply a controllable force to the target surface, and one or more processors including e.g., a controller for the ultrasound device, a processor for a computer or the like that receives ultrasound data from the ultrasound device, and so forth. The one or more processors may be configured to acquire a first data stream from the ultrasound transducer and a second data stream from the force sensor. The one or more processors may be further configured to drive the force controller to apply a predetermined force signature to the target surface of the object, and to obtain synchronized data from the force sensor and the ultrasound transducer based on artifacts from the predetermined force signature by processing the data from the force sensor and the ultrasound transducer to align force data from the force sensor with an integrated optical flow velocity within an ultrasound video image from the ultrasound transducer. The one or more processors may be further configured to adjust an image from the ultrasound transducer according to the synchronized data from the force sensor and the ultrasound transducer.

According to various embodiments described herein, the force sensor may include two or more load cells positioned to measure a force distribution across the ultrasound transducer when placed for use on the target surface. The predetermined force signature may include two or more high frequency variations to the instantaneous contact force between the ultrasound transducer and the target surface, or any other force signature providing detectable artifacts in ultrasound and force data from the device. In one aspect, the one or more processors are configured to adjust the image from the ultrasound transducer by using quantitative measures of elasticity to normalize the image to an adjusted image representative of a uniform contact force across a contact region between the ultrasound transducer and the target surface, e.g., with a distributed load totaling one Newton or some other suitable overall contact force.

FIG. 12 shows a generalized ultrasound workflow using acquisition states such as the distributed force described above. In general, an ultrasound probe such as any of the devices described herein may capture image data and acquisition state data during an ultrasound scan. In one aspect, this data may be fed directly to a user such as an ultrasound technician during a scan. For example, ultrasound images and/or acquisition state data may be displayed on a display of a computer or the like while a scan is being performed.

In another aspect, machine intelligence may be applied in a variety of manners to augment a scanning process. For example, acquisition state data concerning, e.g., a pose of the ultrasound probe may be used to create a graphical representation of a scanner relative to a target, and the graphical representation may be depicted on the display showing a relative position of the ultrasound probe to the target in order to provide visual feedback to the user concerning orientation. As another example, contact force, including a distributed load or contact force, may be displayed as numerical values, and/or an ultrasound image with a normalized contact force may be rendered for viewing by the user during the scan. As another example, the contact force may be provided as a visual or audio indication on the probe, either as quantitative information or as user feedback concerning correct probe positioning, orientation, force vector(s) and so forth. In another aspect, a desired acquisition state may be determined (e.g., provided by a user to the computer), and the machine intelligence may create instructions for the user that can be displayed during the scan to steer the user toward the desired acquisition state. This may be a state of diagnostic significance, and/or a previous acquisition state from a current or historical scan. In another aspect, the desired acquisition state may be transmitted as control signals to the ultrasound probe. For example, control signals for an instantaneous contact force may be communicated to a force-controlled ultrasound device such as the device described above. This may also or instead include scanning data parameters such as frequency or array beam formation, steering, focusing, and the like.

In addition, the capability of capturing a multi-factor acquisition state including, e.g., contact force or a distributed contact force and position permits enhancements to analysis and diagnostic use of an ultrasound system. For example, analysis may include elastography, image normalization, three-dimensional reconstruction (e.g., using normalized images), volume and/or shape analysis, and the like. Similarly, diagnostics may be improved, or new diagnostics created, based upon the resulting improved ultrasound images as well as normalization of images and accurate assessment of an acquisition state. All such uses of an ultrasound system having acquisition state capabilities, feedback control capabilities, and machine intelligence as contemplated herein and are intended to fall within the scope of this disclosure.

It will be appreciated that many of the above systems, devices, methods, processes, and the like may be realized in hardware, software, or any combination of these suitable for the data processing, data communications, and other functions described herein. This includes realization in one or more microprocessors, microcontrollers, embedded microcontrollers, programmable digital signal processors or other programmable devices or processing circuitry, along with internal and/or external memory. This may also, or instead, include one or more application specific integrated circuits, programmable gate arrays, programmable array logic components, or any other device or devices that may be configured to process electronic signals. It will further be appreciated that a realization of the processes or devices described herein may include computer-executable code created using a structured programming language such as C, an object oriented programming language such as C++, or any other high-level or low-level programming language (including assembly languages, hardware description languages, and database programming languages and technologies) that may be stored, compiled or interpreted to run on one of the above devices, as well as heterogeneous combinations of processors, processor architectures, or combinations of different hardware and software. At the same time, processing may be distributed across devices such as the handheld probe and a remote desktop computer or storage device, or all of the functionality may be integrated into a dedicated, standalone device including without limitation a wireless, handheld ultrasound probe. All such permutations and combinations are intended to fall within the scope of the present disclosure.

In other embodiments, disclosed herein are computer program products comprising computer-executable code or computer-usable code that, when executing on one or more computing devices (such as the controller described above), performs any and/or all of the steps described herein. The code may be stored in a computer memory or other non-transitory computer readable medium, which may be a memory from which the program executes (such as internal or external random access memory associated with a processor), a storage device such as a disk drive, flash memory or any other optical, electromagnetic, magnetic, infrared or other device or combination of devices. In another aspect, any of the processes described herein may be embodied in any suitable transmission or propagation medium carrying the computer-executable code described herein and/or any inputs or outputs from same.

While particular embodiments of the present invention have been shown and described, it will be apparent to those skilled in the art that various changes and modifications in form and details may be made therein without departing from the spirit and scope of this disclosure and are intended to form a part of the invention as defined by the following claims, which are to be interpreted in the broadest sense allowable by law.

1. A device comprising:
an ultrasound transducer;
an enclosure with a handle for the ultrasound transducer;
two force sensors coupled between the enclosure and the ultrasound transducer and positioned to measure an instantaneous contact force at two or more corresponding locations around the ultrasound transducer when the ultrasound transducer is placed against a surface of an object for use; and
a processor configured to calculate a force distribution across the ultrasound transducer based on a first set of signals from the two force sensors.

2. The device of claim 1 wherein the ultrasound transducer includes a transducer array of two or more discrete ultrasound transducers.

3. The device of claim 1 wherein the device is configured to calculate shear wave elasticity in a region of interest within the object, and the processor adjusts the shear wave elasticity based upon the force distribution across the ultrasound transducer.

4. The device of claim 1 wherein the processor is configured to create a two-dimensional image of a transverse plane of the object based on a second set of signals from the ultrasound transducer when placed against the surface of the object for use.

5. The device of claim 4 wherein the processor is configured to normalize the two-dimensional image to a predetermined contact force based upon the force distribution across the ultrasound transducer.

6. The device of claim 5 wherein the processor is configured to normalize the two-dimensional image to the predetermined contact force using quantitative elastography.

7. The device of claim 1 comprising a third force sensor coupled between the enclosure and the ultrasound transducer and positioned to measure a third instantaneous contact force at a third corresponding location around the ultrasound transducer when the ultrasound transducer is placed against the surface of the object for use.

8. The device of claim 7 wherein the force distribution is calculated along a line between the two force sensors, and wherein the third force sensor lies on the line to provide an additional force measurement for improving an accuracy of the force distribution calculated along the line.

9. The device of claim 7 wherein the force distribution is calculated in at least two dimensions, and wherein the third force sensor is positioned away from a line between the two force sensors in order to provide additional measurements off of an axis of the line.

10. The device of claim 1 wherein the two force sensors are positioned within a transverse imaging plane of the ultrasound transducer.

11. A method comprising:
providing an ultrasound transducer coupled to an enclosure by two force sensors at two predetermined positions relative to the ultrasound transducer and the enclosure;
applying a positioning force to the enclosure to contact the ultrasound transducer to a target surface of an object;
measuring a force at each of the two force sensors; and
calculating a force distribution over a contact region between the ultrasound transducer and the target surface based on the two predetermined positions and a signal received from each of the two force sensors.

12. The method of claim 11 further comprising receiving ultrasound signals from the ultrasound transducer and calculating a shear wave elasticity within a transverse plane through the object based on the ultrasound signals.

13. The method of claim 12 further comprising adjusting the shear wave elasticity based on the force distribution across the ultrasound transducer.

14. The method of claim 11 further comprising constructing an ultrasound image in a transverse plane through the object based on signals from the ultrasound transducer.

15. The method of claim 14 wherein the ultrasound transducer includes a transducer array of two or more discrete ultrasound transducers.

16. The method of claim 15 further comprising normalizing a two-dimensional image in the transverse plane to a predetermined contact force based on the force distribution across the ultrasound transducer.

17. The method of claim 16 wherein normalizing the two-dimensional image in the transverse plane to the predetermined contact force includes applying quantitative elastography to normalize the two-dimensional image to an adjusted image representative of a uniform contact force across the contact region.

18. The method of claim 11 wherein the ultrasound transducer is coupled to the enclosure through three or more force sensors.

19. The method of claim 11 wherein the two force sensors lie in a transverse plane for capturing images of the object with the ultrasound transducer.

20. A computer program product for processing ultrasound signals from an ultrasound transducer coupled to an enclosure through two force sensors at two predetermined positions, the computer program product comprising computer executable code embodied in a non-transitory computer readable medium that, when executing on one or more computing devices, performs the steps of:
receiving a force signal from each of the two force sensors;
calculating a force distribution over a contact region between the ultrasound transducer and a target surface of an object based on the two predetermined positions and a signal received from each of the two force sensors; and
adjusting an ultrasound image of the object acquired with the ultrasound transducer based on the force distribution over the contact region.

21-40. (canceled)
